(19) United States
(12) Patent Application Publication
(10) Pub. No.: US 2015/0209177 A1
(43) Pub. Date: Jul. 30, 2015

(54) CONTAINER CLOSURE SYSTEM WITH INTEGRAL ANTIMICROBIAL ADDITIVES

(71) Applicant: ALLERGAN, INC., IRVINE, CA (US)

(72) Inventors: Ayako Hasegawa, Aliso Viejo, CA (US); Sai Shankar, Irvine, CA (US); Kunal Jariwala, Orange, CA (US); Melissa Gulmezian, Irvine, CA (US); Ramakrishnan Srikumar, Aliso Viejo, CA (US)

(21) Appl. No.: 14/117,338

(22) PCT Filed: May 11, 2012
(86) PCT No.: PCT/US12/37500

§ 371 Date: Nov. 12, 2013

Related U.S. Application Data
(60) Provisional application No. 61/486,056, filed on May 13, 2011.

(51) Int. Cl. A61F 9/00

(52) U.S. Cl. CPC A61F 9/0008

(57) ABSTRACT

A container-closure system includes a sterile vessel configured to store a preservative-free therapeutic agent. A polymeric applicator is fluidly coupled to the vessel through which the therapeutic agent is dispensed. Surfaces of the applicator that are susceptible to contamination during dispensing of the therapeutic agent comprise one or more antimicrobial additives which provide antimicrobial efficacy at the applicator surfaces.

### RELATED PATENT DOCUMENTS

This application claims the benefit of U.S. Provisional Patent Application Ser. No. 61/486,056, filed May 13, 2011, to which priority is claimed pursuant to 35 U.S.C. §119(e) and which is hereby incorporated herein by reference.

### SUMMARY

Embodiments of the disclosure are directed to container-closure systems and drug delivery systems fabricated from a polymeric material that incorporates or is coated with antimicrobial additives at surfaces that are susceptible to microbial contamination during repeated use. According to some embodiments, a container-closure system for dispensing a preservative-free therapeutic agent includes a sterile vessel configured to store the preservative-free therapeutic agent, and a polymeric applicator fluidly coupled to the vessel and through which the therapeutic agent is dispensed. Surfaces of the applicator that are susceptible to contamination during dispensing of the therapeutic agent comprise one or more antimicrobial additives which provide antimicrobial efficacy at the applicator surfaces.

Some embodiments are directed to a container-closure system for dispensing a partially-preserved or preserved therapeutic agent. Such embodiments include a polymeric vessel configured to store the partially-preserved or preserved therapeutic agent, and a polymeric applicator fluidly coupled to the vessel and through which the therapeutic agent is dispensed. Surfaces of the applicator and the vessel that are susceptible to contamination during dispensing of the therapeutic agent comprise one or more antimicrobial additives which provide antimicrobial efficacy at the applicator and vessel surfaces.

In accordance with other embodiments, a method for dispensing a preservative-free therapeutic agent involves storing the preservative-free therapeutic agent within a polymeric vessel, where the vessel is fluidly coupled to a polymeric applicator through which the therapeutic agent is dispensed. The method also involves providing antimicrobial efficacy at surfaces of the applicator that can be contaminated during dispensing of the therapeutic agent. Providing antimicrobial efficacy may involve providing antimicrobial efficacy at the applicator surfaces for at least a predetermined number of days and without fouling the therapeutic agent.

According to further embodiments, a method for dispensing a partially-preserved or preserved therapeutic agent involves storing the partially-preserved or preserved therapeutic agent within a polymeric vessel. The vessel is fluidly coupled to a polymeric applicator through which the therapeutic agent is dispensed. The method also involves providing antimicrobial efficacy at surfaces of the applicator and the vessel that can be contaminated during dispensing of the therapeutic agent. Providing antimicrobial efficacy may involve providing antimicrobial efficacy at the applicator surfaces for at least a predetermined number of days and without fouling the therapeutic agent.

Some embodiments of the present invention include the following:
1. A container-closure system for dispensing a preservative-free therapeutic agent, comprising:

a sterile vessel configured to store the preservative-free therapeutic agent; and

a polymeric applicator fluidly coupled to the vessel and through which the therapeutic agent is dispensed, wherein surfaces of the applicator that are susceptible to contamination during dispensing of the therapeutic agent comprise one or more antimicrobial additives which provide antimicrobial efficacy at the applicator surfaces.
2. The system according to paragraph 1, wherein the container-closure system is configured for ophthalmic administration of drugs.
3. The system according to paragraph 1, wherein the container-closure system is configured to dispense a plurality of single doses of the therapeutic agent in the form of an ophthalmic solution, emulsion or suspension.
4. The system according to paragraphs 1-3, wherein:

the applicator comprises a dropper having a cap and a tip;

the antimicrobial additives are distributed on the tip of the dropper and interior surfaces of the cap that are exposed during dispensing of the therapeutic agent; and

the antimicrobial additives have an antimicrobial effect when in physical contact with the therapeutic agent.
5. The system according to paragraphs 1-4, wherein the applicator is inseparable from the vessel.
6. The system according to paragraphs 1-5, further comprising a unidirectional valve fluidly coupled between the vessel and the applicator.
7. The system according to paragraphs 1-6, wherein the applicator surfaces comprise a polymeric material impregnated or embedded with the one or more antimicrobial additives.
8. The system according to paragraph 7, wherein the one or more antimicrobial additives is selected from the group consisting of silver select, ion pure IPL, biosafe, a combination of biosafe and ion pure IPL, IRGAGUARD® F3000, Triclosan, zinc omadine, zinc ion, cupper ion, cerium ion, GOLDSHIELD®, AEGIS™ antimicrobial, and PEI-TCS polymers, alone or in any combination thereof.
9. The system according to paragraphs 1-6, comprising a coating or film applied to the applicator surfaces, the coating or film comprising the one or more antimicrobial additives.
10. The system according to paragraph 9, wherein the one or more antimicrobial additives is selected from the group consisting of silver nanoparticles, biosafe, IRGAGUARD® F3000, Triclosan, zinc omadine, zinc ion, cupper ion, cerium ion, GOLDSHIELD®, AEGIS™ antimicrobial, PEI-TCS polymers, protamine sulfate and chlorhexidine, alone or in any combination thereof.
11. The system according to paragraphs 1-10, wherein the one or more antimicrobial additives provide antimicrobial efficacy for at least a predetermined number of days.
12. The system according to paragraphs 1-11, wherein the one or more antimicrobial additives provide antimicrobial efficacy without fouling the therapeutic agent.
13. The system according to paragraphs 1-12, wherein the applicator surfaces comprise a plurality of antimicrobial additives, at least some of the plurality of the antimicrobial additives differing in terms of breadth of a spectrum of microorganisms covered or a rate at which microorganisms are killed.
14. The system according to paragraphs 1-13, wherein the therapeutic agent is in solution, emulsion or suspension form, and the therapeutic agent is selected from the group consisting of bimatoprost, brimonidine, timolol, cyclosporine, gatifloxacin, ocufloxacin, prednisolone, carnitine and ketorolac.
15. The system according to paragraphs 1-14, wherein the vessel and the applicator are formed of one or more polymers selected from the group consisting of low-density polyethylene, high-density polyethyelene, and high-impact polystyrene.
16. A container-closure system for dispensing a partially-preserved or preserved therapeutic agent, comprising:

a polymeric vessel configured to store the partially-preserved or preserved therapeutic agent; and

a polymeric applicator fluidly coupled to the vessel and through which the therapeutic agent is dispensed, wherein surfaces of the applicator and the vessel that are susceptible to contamination during dispensing of the therapeutic agent comprise one or more antimicrobial additives which provide antimicrobial efficacy at the applicator and vessel surfaces.
17. The system according to paragraph 16, wherein:

the applicator comprises a dropper having a cap and a tip;

the antimicrobial additives are distributed on the tip of the dropper and interior surfaces of the cap that are exposed during dispensing of the therapeutic agent; and

the antimicrobial additives have an antimicrobial effect when in physical contact with the therapeutic agent.
18. The system according to paragraphs 16 or 17, wherein the antimicrobial additives are distributed on an interior wall of the vessel, the antimicrobial additives providing antimicrobial efficacy at vessel wall surfaces that become exposed as the volume of therapeutic agent within the vessel is reduced due to repeated dispensing over time.
19. A method for dispensing a preservative-free therapeutic agent, comprising:

storing the preservative-free therapeutic agent within a polymeric vessel, the vessel fluidly coupled to a polymeric applicator through which the therapeutic agent is dispensed; and

providing antimicrobial efficacy at surfaces of the applicator that can be contaminated during dispensing of the therapeutic agent.
20. The method according to paragraph 19, wherein providing antimicrobial efficacy comprises providing antimicrobial efficacy at the applicator surfaces for at least a predetermined number of days and without fouling the therapeutic agent.
21. A method for dispensing a partially-preserved or preserved therapeutic agent, comprising:

storing the partially-preserved or preserved therapeutic agent within a polymeric vessel, the vessel fluidly coupled to a polymeric applicator through which the therapeutic agent is dispensed; and

providing antimicrobial efficacy at surfaces of the applicator and the vessel that can be contaminated during dispensing of the therapeutic agent.
22. The method according to paragraph 21, wherein providing antimicrobial efficacy comprises providing antimicrobial efficacy at the applicator surfaces for at least a predetermined number of days and without fouling the therapeutic agent.

These and other features can be understood in view of the following detailed discussion and the accompanying drawings.

### BRIEF DESCRIPTION OF THE DRAWINGS

FIG. 1 shows a container-closure system or drug delivery system (collectively referred to hereinafter as a “container-closure system” for simplicity) formed of polymeric material that includes antimicrobial additives in accordance with various embodiments;

FIG. 2 illustrates a container-closure system formed of a polymeric material having selected surfaces treated with one more antimicrobial additives in accordance with other embodiments of the disclosure;

FIGS. 3A-3D are diagrams that represent cross-sectional views of a portion of the vessel enclosures shown in FIGS. 1 and 2 in accordance with embodiments of the disclosure;

FIGS. 4-6 illustrate different types of container-closure systems configured to store a therapeutic agent having one or more preservatives and formed of a polymeric material having selected surfaces treated with one more antimicrobial additives in accordance with other embodiments of the disclosure;

FIG. 7 illustrates a method of dispensing a preservative-free therapeutic agent to target tissue of the body in accordance with embodiments of the disclosure; and

FIG. 8 illustrates a method of dispensing a preserved or partially-preserved therapeutic agent to target tissue of the body in accordance with embodiments of the disclosure.

### DISCLOSURE

Embodiments of the disclosure are generally directed to container-closure and drug delivery systems fabricated from a polymeric material and comprising antimicrobial additives provided at selected surfaces that are susceptible to microbial contamination during use. In some embodiments, selected surfaces of a polymeric container-closure or drug delivery system are impregnated with antimicrobial additives to prevent microbial growth at the selected surfaces that are susceptible to microbial contamination during use. Other embodiments are directed to container-closure and drug delivery systems fabricated from polymeric material and include a coating or film containing antimicrobial additives applied to selected surfaces that are susceptible to microbial contamination during use. According to some embodiments, the container-closure or drug delivery systems are configured for dispensing multiple single doses of a therapeutic agent in the form of a solution, emulsion or suspension. One or more antimicrobial additives of the container-closure or drug delivery system are selected to provide antimicrobial efficacy at selected surfaces of the system exposed during repeated use for a predetermined duration of time, such as a predetermined number of days (e.g., one month).

Various embodiments of the disclosure are directed to use of antimicrobial additives in plastic resins, coatings, and films for application in ophthalmic container-closure systems and drug-delivery systems for purposes of reducing the risk of microbial contamination during repeated use. According to various embodiments, low levels of antimicrobial additives (stand-alone or in combinations thereof) are impregnated, embedded, surface treated or coated in/on surfaces of plastic ophthalmic container-closure/drug-delivery systems that are susceptible to microbial contamination during repeated use.

According to some embodiments, antimicrobial additives are provided at selected surfaces of plastic container-closure systems or drug delivery systems, such as on surfaces of an ophthalmic dropper (e.g., the tip and/or interior of the dropper cap) which are susceptible to microbial contamination. A variety of ophthalmic multi-dose container-closure systems and drug delivery systems can benefit from inclusion of antimicrobial surface protection according to embodiments of the disclosure, including those that contain unpreserved, partially preserved, and preserved ophthalmic products. Embodiments of the disclosure can be used in conjunction with multi-use preservative-free technologies such as, but not limited to, unidirectional valve or filter systems that prevent re-entry of product into the main bladder of the dropper during repeated use.

Embodiments of the disclosure are of particular importance for ophthalmic products, since impregnating the antimicrobials in the plastic (or covering the plastic with antimicrobials) of the container-closure/drug delivery system can help mitigate issues that could otherwise interfere with the safety and commercial success of the drug product. It is a known concern that conventional preservatives in solution at high concentrations can interfere with the safety and commercial success of a product due to corneal and ocular toxicity of these conventional preservatives. By impregnating, embedding or surface treating the antimicrobials in the plastic, embodiments of the disclosure offer the potential to provide antimicrobial protection without conventional preservatives. Embodiments of the disclosure serve to alleviate possible microbial contamination of ophthalmic container-closure/drug delivery system at the exposed surfaces during repeated use.

Turning now to FIG. 1, there is illustrated a container-closure system or drug delivery system formed of polymeric material that includes antimicrobial additives in accordance with various embodiments. For purposes of simplicity, the embodiments illustrated in the figures will be described as container-closures, although it is understood that such embodiments are also applicable to drug delivery systems of various types. The container-closure 100 shown in FIG. 1 is preferably configured to dispense a preservative-free therapeutic agent. It is noted that the container-closure embodiment depicted in FIG. 1 and other figures can also be configured for dispensing therapeutic agents that include a preservative, representative examples of which are described hereinbelow.

The container-closure 100 includes a vessel 101 having an enclosure 102 configured to store a preservative-free therapeutic agent 106 therein. The vessel enclosure 102 is sterile in embodiments where the enclosure 102 is implemented to store a preservative-free therapeutic agent 106. The vessel 101 is fluidly coupled to a polymeric applicator 104 through which the therapeutic agent is dispensed through an orifice 105. Surfaces of the applicator 104 that are susceptible to microbial contamination are provided with one or more antimicrobial additives which provide antimicrobial efficacy at the applicator surfaces. For example, outer surfaces 103 of the applicator 104 which are likely to come into contact with a microbial contaminant are provided with antimicrobial additives. Typical examples of microbial contaminating elements include a body surface or mucous of a user, structures upon which the container-closure 100 rests, and the ambient environment surrounding the container-closure 100.

In some container-closure configurations, it has been found that an inner wall 109 of the channel 107 can become susceptible to microbial growth, and is preferably provided with antimicrobial additives to prevent such growth. For example, it is preferable that antimicrobial additives be included at the surfaces of the inner wall 109 between the unidirectional valve 110 and the orifice 105 of the applicator 104. The surface of the unidirectional valve 110 adjacent the orifice 105 is also preferably treated to include antimicrobial additives.

The container-closure 100 includes a channel 107 that fluidly couples the vessel 101 with the applicator 104. According to some embodiments, the channel 107 includes a unidirectional valve 110, which may optionally include a filter. The unidirectional valve 110 is configured to allow the therapeutic agent 106 contained within the vessel 101 to pass through to the applicator 104, but prevents re-entry of the therapeutic agent 106 and other fluids or contaminants into the vessel 101. Various types of valves can be implemented to provide unidirectional flow of fluid from the vessel 101 to the applicator 104, including the Novelia valve available from Rexam and the valve system of the Opthalmic Squeeze Dispenser available from Aptar Pharma, for example.

In some embodiments, the container-closure 100 is configured to dispense a single dose of the therapeutic agent 106 on a repeated basis over a predetermined duration of time.

For example, the container-closure 100 can be configured to dispense single doses of the therapeutic agent 106 each day for a month. According to some embodiments, the container-closure 100 is configured to dispense a predetermined volume of the therapeutic agent 106 as a single dose. In such embodiments, the unidirectional valve 110 can be configured to regulate the volume of the therapeutic agent 106 so that a metered dose of the therapeutic agent 106 is dispensed during each application. Suitable precision metering valves are available from Rexam, for example. Also, various available spring-loaded unidirectional valves can be used that open during actuation to deliver a single dose of drug product. After actuation, the valve returns to its original position and seals the opening.

Referring now to FIG. 2, there is illustrated a container-closure 200 formed of a polymeric material having selected surfaces treated with one more antimicrobial additives in accordance with other embodiments of the disclosure. According to the embodiment shown in FIG. 2, the container-closure 200 includes a vessel 201 having an enclosure 202 configured to store a preservative-free therapeutic agent 206. As in the embodiment shown in FIG. 1, the vessel 201 is preferably a sterile container that maintains sterility of the therapeutic agent during repeated use of the container-closure 200. The container-closure 200 includes an applicator 204 fluidly coupled to the vessel 201 via channel 207 which preferably incorporates a unidirectional valve 210 of the type previously described. The unidirectional valve 210 can be configured to dispense a metered dose of the therapeutic agent 206 during each application.

The applicator 204 has a generally tapered shape that is appropriately dimensioned for dispensing a therapeutic agent 206 to a localized portion of a user's body, such as the eyes, nostrils, and ears. For example, the container-closure 200 can be implemented to contain a preservative-free ophthalmic therapeutic agent and the applicator 204 may be configured to enable a user to dispense the ophthalmic therapeutic agent multiple times to the eyes over an extended period of time, such as one month.

The outwardly extending applicator 204 shown in FIG. 2 defines a dropper through which the therapeutic agent 206 can be dispensed to a particularized location of the body with relative precision. The container-closure 200 shown in FIG. 2 includes a cap 215 which is configured to releasably engage a distal portion of the dropper 204. When properly positioned at the distal portion of the dropper 204, a seal is formed between the cap 215 and the dropper 204. Depending on the nature of the therapeutic agent 206 and the application of use, the seal can be implemented to provide a desired degree of sealing (e.g., fluid-tight, air-tight, or mechanically tight). The cap 215 is preferably coupled to the enclosure 202 of the vessel 201 via a tether 213, which may be formed during molding of the container-closure 200.

According to embodiments in which the vessel 201 and valve 210 are configured to maintain sterility of the therapeutic agent 206, selected surfaces of the dropper 204 includes one or more antimicrobial additives. In the representative example shown in FIG. 2, the dropper 204 includes an outer surface 211 that is susceptible to microbial contamination during use. In some embodiments, only a distal portion of the dropper 204 includes antimicrobial additives (e.g., the last 25-50% of the dropper length). In other embodiments, the entire outer surface of the dropper 204 can be provided with antimicrobial additives. As previously discussed, it is been found beneficial to provide antimicrobial protection for all or a portion of an inner wall 209 of the channel 207. Additionally, surfaces of the cap 215 that are susceptible to microbial contamination are also provided with antimicrobial additives. In some embodiments, a satisfactory level of antimicrobial protection can be achieved by providing antimicrobial additives at the inner surface 217 of the cap 215. The outer surface 216 may also contain antimicrobial additives.

A variety of therapeutic agents can be dispensed using container-closure systems implemented in accordance with embodiments of the disclosure. A non-limiting, non-exhaustive list of such therapeutic agents includes bimatoprost, brimonidine, timolol, cyclosporine, gatifloxacin, ocufloxacin, prednisolone, carnitine and ketorolac. The systems implemented in accordance with embodiments of the disclosure are not limited to delivery of preservative-free therapeutic agents, but can also be applied to delivery of preserved therapeutic agents.

Referring now to FIGS. 3A-3D, various diagrams are shown that represent cross-sectional views of a portion of the vessel enclosures 102/202 respectively shown in FIGS. 1 and 2. These sectional views may represent relatively thin material portions of the enclosures 102/202 (e.g., sidewalls) or near-surface regions of thicker portions of the enclosures 102/202. In FIG. 3A, polymeric material section 300 is shown impregnated with one or more antimicrobial additives through the entire cross section. The antimicrobial additives may be distributed substantially uniformly within the section 300, or as shown here, be non-uniformly distributed (e.g., greater concentration of antimicrobial near the outer surface 302). Various techniques can be used to impregnate polymeric material with one or more antimicrobial additives, representative examples of which are disclosed in U.S. Published Application No. 2005/0142200, which is incorporated herein by reference.

In FIG. 3B, polymeric material section 304 includes two layers 306 and 308. Layer 306 represents the portion of section 304 that is substantially devoid of antimicrobial additives. In some embodiments, layer 308 represents the portion of section 304 which is impregnated with one or more antimicrobial additives. In other embodiments, layer 308 represents the portion of section 304 within which one or more antimicrobial additives are embedded within the polymeric material section 304. In further embodiments, layer 308 represents the portion of section 304 which is treated with a coating or film containing one or more antimicrobial additives. In each of these embodiments, the antimicrobial additives prevent microbial growth on the surface 310 of section 304 which is susceptible to microbial contamination.

FIG. 3C shows an embodiment of polymeric material section 314 which includes a multiplicity of antimicrobial protection layers 318a-318n and a portion 316 which is substantially devoid of an antimicrobial additive. Each of the layers 318a-318n comprises one or more antimicrobial additives. The antimicrobial layers cooperate to provide continuous antimicrobial efficacy across of surface 320 for a predetermined duration of time (e.g., one month). The antimicrobial efficacy of the layers 318a-318n typically differs from one another, but cooperate to provide sustained antimicrobial efficacy across the surface 320 of section 314 for the required duration. The layers of 318a-318n can differ from one another in terms of thickness, porosity, hydrophobicity, antimicrobial additive activity, concentration, composition, rate of effectiveness, duration of effectiveness, and breadth of spectrum of microorganisms covered by the antimicrobials, among other properties. The layers 318a-318n can be formed using an impregnation, embedding, or coating technique, or a combination of these techniques. For example, one layer can be formed using an impregnation technique, while an adjacent layer can be formed by application of a coating or film.

The polymeric material section 324 shown in FIG. 3D is similar to that shown in FIG. 3B. Section 324 includes layer 326, which represents a portion of section 324 substantially devoid of an antimicrobial additive, and layer 328, which includes one or more antimicrobial additives that prevent microbial growth on the surface 330 that is susceptible to microbial contamination. Section 324 further includes a permeable top layer 329 which serves to moderate antimicrobial activity across the surface 330 to achieve a desired level of antimicrobial efficacy without fouling the therapeutic agent that contacts the polymeric material section 324. In some embodiments that include a multiplicity of antimicrobial protection layers, such as in the embodiment shown in FIG. 3C, a permeable layer 329 can be provided between adjacent antimicrobial protection layers to aid in moderating antimicrobial activity across the surface 330 of section 324.

As was previously discussed, selected surfaces of a polymeric container-closure system can be impregnated (or embedded) with antimicrobial additives and/or covered with a coating or film containing antimicrobial additives to prevent microbial growth at the selected surfaces which are susceptible to microbial contamination during use. It was found that several antimicrobial additives evaluated by the inventors are relatively versatile, in that they can be incorporated into or onto polymeric material using a variety of incorporation techniques, such as an impregnation technique, an embedding technique, a coating technique or a surface treatment technique. It was further found that some antimicrobial additives are less versatile than others, in that such antimicrobials can be incorporated into or onto polymeric material using a limited number of incorporation techniques.

It was determined that the following antimicrobial additives can be impregnated in (or embedded within) polymeric material suitable for fabricating container-closure systems according to various embodiments of the disclosure: silver select, ion pure IPL, biosafe, a combination of biosafe and ion pure IPL, IRGAGUARD® F3000, Triclosan, zinc omadine, zinc ion, cupper ion, cerium ion, GOLDSHIELD®, AEGIS™ antimicrobial, and PEI-TCS polymers, alone or in any combination thereof.

It was determined that the following antimicrobial additives can be incorporated in a coating that can be applied to polymeric material suitable for fabricating container-closure systems according to other embodiments of the disclosure: silver nanoparticles, biosafe, IRGAGUARD® F3000, Triclosan, zinc omadine, zinc ion, cupper ion, cerium ion, GOLDSHIELD®, AEGIS™ antimicrobial, PEI-TCS polymers, protamine sulfate and chlorhexidine, alone or in any combination thereof.

### EXAMPLE #1

Antimicrobial efficacy of individual and combinations of selected antimicrobials impregnated or surface treated in various plastic polymers was determined using the modified American National Standards Institute (ANSI) JIS Z 2801 test against a broad spectrum of microorganisms. An example of these results is shown in Table 1 below.

The standard test, JIS Z 2801, is utilized in order to test log-fold reduction of microorganisms applied onto plastic plaques treated with antimicrobials. A broad spectrum of microorganisms, bacteria, yeast and mold, are tested in order to assess the efficacy of the antimicrobial-treated plastic. High concentration of microorganisms (10⁶) are aliquoted onto plaques and covered with a 40 mm² cover slip in order to evenly distribute the drop and ensure that the drop contacts the surface. At specified time points, the plaques are neutralized with a qualified neutralizer, rinsed thoroughly, and serial dilutions of the rinsate are performed in order to obtain colonies in a countable range. Bacteria are then plated onto Soybean Casein Digest Agar (SCDA) plates, and yeast and mold are plated onto Sabouraud Dextrose Agar (SDA) plates for counting. The microbial log-fold reduction is calculated by comparing microorganisms recovered from the treated plaques to the control plaques.

Table 1 below provides testing results showing the efficacy of microbial log-fold reduction on different polymer types treated with a variety of antimicrobials.

TABLE 1

| | Active Concentration | | |
|---|---|---|---|
| Material | Range tested (%)^{a} | Polymer Types | Efficacy |
| Silver | 1~2 | LDPE, HDPE | Broad spectrum (Staph |
| Select | | and HIPS^{b} | aureus, Pseudomonas |
| Ion Pure | 1~2 | | aeruginosa, Escherichia |
| BioSafe | 0.5~1 | | coli, Candida albicans, |
| IonPure, | 1~2 and 0.5~1, | | Aspergilus brasiliensis) |
| BioSafe | respectively | | |
| Combo | | | |

^{a}For Silver Select and Ion Pure the concentration listed is of silver.
^{b}LDPE = low-density polyethylene,
HDPE = high-density polyethyelene
HIPS = high-impact polystyrene.

### EXAMPLE #2

### Sample Treatment

### 7 Day Wash Dry Cycle (7d WDC):

1. WDC testing was performed in order to mimic repeated wetting and drying of container closure parts that would occur through patient use. A JIS study was carried out after WDC testing in order to assess the effect of wetting and drying on the efficacy of antimicrobial-treated plastic.
2. The plaques were washed 8 times a day for 7 days. In between each day, the plaques were laid out for drying. Total washes=56 times.
   a. The plaques were placed in a plastic box and approximately 350 mL of Saline Tween 80 was added in order to carry out the washing. During each washing, the box was shaken and each plaque was ensured to be completely soaked in Saline Tween 80 solution.
   b. Following the washing, Saline Tween 80 solution was completely decanted.
   c. Steps 2a-2b were repeated 8 times consecutively and following the 8^{th} washing and decanting, the plaques were placed on a wire rack at room temperature to allow for complete drying.
   d. Washing and drying cycles described in steps 2a-2c were carried out for a total of 7 days.

3 Day Immersion (3d I):
1. Plaques were immersed in a solution continuously for 3 days in order to assess the effect of stringent, prolonged wet conditions on the plaques. A JIS study was carried out after the immersion in order to assess the effect of immersion on the efficacy of antimicrobial-treated plastic.
2. The plaques were immersed completely for a total of 3 days. After the immersion, the plaques were laid out for drying.
   a. The plaques were placed in a container and approximately 350 mL of Saline Tween 80 was added and ensured that each plaque was completely submerged in the solution. After adding the solution, the container was shaken vigorously.
   b. The plaques were held in the container, completely immersed, for 3 continuous days at room temperature.
   c. Following 3 days of immersion, the Saline Tween 80 solution was decanted and the plaques were placed on a wire rack at room temperature to allow for complete drying.

Table 2 below provides the testing results showing the efficacy of microbial log-fold reduction on antimicrobial-treated HDPE plastic.

TABLE 2

| | Active | | |
|---|---|---|---|
| | Concentration | Polymer | |
| Material | Range tested (%) | Types | Efficacy |
| Ion Pure | 2 | HDPE | Broad spectrum |
| Silver Select | 5 | | coverage |
| Zinc Omadine | 0.05-0.5 | | |

^{a}Staphylococcus aureus, Pseudomonas aeruginosa, Escherichia coli, Candida albicans, and Aspergillus brasiliensis

Table 3 below provides the testing results showing the efficacy of microbial log-fold reduction on antimicrobial-treated plastic, after wash dry cycles, or immersion.

TABLE 3

| | Active | | |
|---|---|---|---|
| | Concentration | Polymer | |
| Material | Range tested (%) | Types | Efficacy |
| Ion Pure - 7d WDC^{b} | 2 | HDPE | No efficacy |
| Silver Select - 7d WDC | 2 | | No efficacy |
| Zinc Omadine - 7d WDC | 0.5 | | Effective^{a} |
| Ion Pure - 3d I^{c} | 2 | | No efficacy |
| Zinc Omadine - 3d I | 0.5 | | Effective |

^{a}Efficacy demonstrated for Staphylococcus aureus, and Pseudomonas aeruginosa
^{b}7 Day Wash Dry Cycle
^{c}3 Day Immersion

Depending on the nature of the antimicrobial, the nature of the plastic, the nature of the product and/or the spectrum of microorganism that coverage is required, the antimicrobials listed here may be used alone or in combination. The antimicrobial activity may require the leaching of the impregnated or surface treated antimicrobial agent from the plastic or be restricted to activity on the surface of the plastic or may require both mechanisms. Based on the dynamics of kill, a fast acting but narrower spectrum antimicrobial and a slower acting but broader spectrum antimicrobial may be combined for enhanced antimicrobial coverage. The nature of the polymer and/or product may dictate either one antimicrobial or a combination for optimal antimicrobial efficacy. The need to enhance the spectrum of coverage to gram-positive and gram-negative bacteria, yeasts and molds typically dictates the choice of antimicrobials, alone or in combination.

FIGS. 4-6 illustrate different types of container-closure systems that are configured to store a therapeutic agent having one or more preservatives and formed of a polymeric material having selected surfaces treated with one or more antimicrobial additives in accordance with various embodiments of the disclosure. It has been found that container-closure systems configured to dispense preserved or partially-preserved therapeutic agents can become fouled by microorganisms over time due to microbial growth on system surfaces not adequately protected by the preservatives. Unchecked microbial growth in such container-closure systems can decrease the effectiveness of the preservatives over time. Inclusion of antimicrobial additives at selected surfaces of the container-closure systems that are susceptible to microbial contamination can reduce the risk of contaminating or fouling of the therapeutic agent.

Referring now to FIG. 4, there is shown a container-closure system 400 configured to store a therapeutic agent provided with a preservative in accordance with various embodiments. The therapeutic agent is stored within a vessel 401 formed as a polymeric enclosure 402. An applicator 404 is fluidly coupled to the vessel 401 via a fluid channel. In the embodiment shown in FIG. 4, the fluid channel typically does not include a unidirectional valve (but may include such a valve in accordance with various embodiments). The fluid channel may incorporate a filter. In the embodiment shown in FIG. 4, the applicator 404 has a generally tapering dropper 406 with a tip having an orifice 405 through which the therapeutic agent is dispensed to a target location of the body, such as the eyes, nostrils, years, or other portion of the body. The container-closure system 400 includes a removable cap 415 which can be screwed on and off of a thread arrangement provided on a base of the applicator 404 or an upper portion of the vessel 401.

The container-closure system 500 shown in FIG. 5 is configured to store a therapeutic agent provided with a preservative in accordance with various embodiments. The therapeutic agent is stored within a vessel 501 formed as a polymeric enclosure 502. The arrows pointing to the vertical dotted lines along the sides of the vessel enclosure 502 illustrate how the vessel 501 can be deformed when squeezed by a user. It is noted that other embodiments of a container-closure system described herein can be implemented with a squeezable vessel. Alternatively, or in addition, a pump mechanism can be implemented to facilitate metered or unmetered dispensing of a therapeutic agent contained within the vessel.

An applicator 504 is fluidly coupled to the vessel 501 via a fluid channel, which need not include a unidirectional valve. The applicator 504 in FIG. 5 includes a relatively short and tapered spout 506 with an orifice 505 through which the therapeutic agent is dispensed to a target location of the body, such as the eyes, nostrils, ears, or other portion of the body. The container-closure system 500 includes a detachable cap 515 which is tethered at a base of the applicator 504.

FIG. 6 shows a container-closure system 600 configured to store a therapeutic agent provided with a preservative in accordance with various embodiments. The therapeutic agent is stored within a vessel 601 formed as a polymeric enclosure 602. An applicator 604 is fluidly coupled to the vessel 601 via a fluid channel, which can include or exclude a unidirectional valve. The applicator 604 in FIG. 6 includes a conical dropper 606 with an orifice 605 through which the therapeutic agent is dispensed to a target location of the body. The container-closure system 600 preferably includes a detachable cap (not shown) that can be tethered to or separable from the applicator 604 or vessel enclosure 602.

In some embodiments, only surfaces of the applicator 404/504/604 that are susceptible to microbial contamination during repeated use are fabricated to include one or more antimicrobial additives. These applicator surfaces include at least an outer surface of the dropper 406/606 or spout 506 and, optionally, an inner wall of the dropper 406/606 or spout 506. In other embodiments, only surfaces of the applicator 404/504/604 and the cap 415/515 that are susceptible to microbial contamination during repeated use are fabricated to include one or more antimicrobial additives. These surfaces include the outer and, optionally, an inner wall of the dropper 406/606 or spout 506, and at least an inner surface of the cap 415/515.

According to further embodiments, all or a portion of an inner wall of the vessel enclosure 402/502/602 can be fabricated to include one or more antimicrobial additives. In such embodiments, the above-described surfaces of the applicator 404/504/604 and cap 415/515 (optionally) are also preferably fabricated to include one or more antimicrobial additives. Provision of one or more antimicrobial additives at the inner surface of the vessel enclosure 402/502/602 provides antimicrobial protection for vessel wall surfaces that are either intermittently or inadequately protected by the preservatives of the therapeutic agent as the volume of the therapeutic agent within the vessel is reduced due to repeated dispensing over time.

FIG. 7 illustrates a method of dispensing a preservative-free therapeutic agent to target tissue of the body in accordance with various embodiments. The method shown in FIG. 7 involves storing 700 a non-preserved therapeutic agent within a polymeric vessel. The polymeric vessel is fluidly coupled 710 to a polymeric applicator through which the therapeutic agent is dispensed. The method of FIG. 7 further involves providing antimicrobial efficacy 720 at surfaces of the applicator that are susceptible to microbial contamination during repeated dispensing over time.

FIG. 8 illustrates a method of dispensing a preserved or partially-preserved therapeutic agent to target tissue of the body in accordance with various embodiments. The method shown in FIG. 8 involves storing 800 a therapeutic agent having a preservative within a polymeric vessel. The polymeric vessel is fluidly coupled 810 to a polymeric applicator through which the therapeutic agent is dispensed. The method of FIG. 8 further involves providing antimicrobial efficacy 820 at surfaces of the applicator and surfaces of the vessel that are susceptible to microbial contamination during repeated dispensing over time.

The foregoing description of the representative embodiments has been presented for the purposes of illustration and description. It is not intended to be exhaustive or to limit the invention to the precise form disclosed. Many modifications and variations are possible in light of the above teaching. Any or all features of the disclosed embodiments can be applied individually or in any combination are not meant to be limiting, but purely illustrative. It is intended that the scope of the invention be limited not with this detailed description, but rather determined by the claims appended hereto.

What is claimed is:

1. A container-closure system for dispensing a preservative-free therapeutic agent, comprising:
a sterile vessel configured to store the preservative-free therapeutic agent; and
a polymeric applicator fluidly coupled to the vessel and through which the therapeutic agent is dispensed, wherein surfaces of the applicator that are susceptible to contamination during dispensing of the therapeutic agent comprise one or more antimicrobial additives which provide antimicrobial efficacy at the applicator surfaces.

2. The system according to claim 1, wherein the container-closure system is configured for ophthalmic administration of drugs.

3. The system according to claim 1, wherein the container-closure system is configured to dispense a plurality of single doses of the therapeutic agent in the form of an ophthalmic solution, emulsion or suspension.

4. The system according to claim 1, wherein:
the applicator comprises a dropper having a cap and a tip;
the antimicrobial additives are distributed on the tip of the dropper and interior surfaces of the cap that are exposed during dispensing of the therapeutic agent; and
the antimicrobial additives have an antimicrobial effect when in physical contact with the therapeutic agent.

5. The system according to claim 1, wherein the applicator is inseparable from the vessel.

6. The system according to claim 1, further comprising a unidirectional valve fluidly coupled between the vessel and the applicator.

7. The system according to claim 1, wherein the applicator surfaces comprise a polymeric material impregnated or embedded with the one or more antimicrobial additives.

8. The system according to claim 7, wherein the one or more antimicrobial additives is selected from the group consisting of silver select, ion pure IPL, biosafe, a combination of biosafe and ion pure IPL, IRGAGUARD® F3000, Triclosan, zinc omadine, zinc ion, cupper ion, cerium ion, GOLDSHIELD®, AEGIS™ antimicrobial, and PEI-TCS polymers, alone or in any combination thereof.

9. The system according to claim 1, comprising a coating or film applied to the applicator surfaces, the coating or film comprising the one or more antimicrobial additives.

10. The system according to claim 9, wherein the one or more antimicrobial additives is selected from the group consisting of silver nanoparticles, biosafe, IRGAGUARD® F3000, Triclosan, zinc omadine, zinc ion, cupper ion, cerium ion, GOLDSHIELD®, AEGIS™ antimicrobial, PEI-TCS polymers, protamine sulfate and chlorhexidine, alone or in any combination thereof.

11. The system according to claim 1, wherein the one or more antimicrobial additives provide antimicrobial efficacy for at least a predetermined number of days.

12. The system according to claim 1, wherein the one or more antimicrobial additives provide antimicrobial efficacy without fouling the therapeutic agent.

13. The system according to claim 1, wherein the applicator surfaces comprise a plurality of antimicrobial additives, at least some of the plurality of the antimicrobial additives differing in terms of breadth of a spectrum of microorganisms covered or a rate at which microorganisms are killed.

14. The system according to claim 1, wherein the therapeutic agent is in solution, emulsion or suspension form, and the therapeutic agent is selected from the group consisting of bimatoprost, brimonidine, timolol, cyclosporine, gatifloxacin, ocufloxacin, prednisolone, carnitine and ketorolac.

15. The system according to claim 1, wherein the vessel and the applicator are formed of one or more polymers selected from the group consisting of low-density polyethylene, high-density polyethylene, and high-impact polystyrene.

16. A container-closure system for dispensing a partially-preserved or preserved therapeutic agent, comprising:
a polymeric vessel configured to store the partially-preserved or preserved therapeutic agent; and
a polymeric applicator fluidly coupled to the vessel and through which the therapeutic agent is dispensed, wherein surfaces of the applicator and the vessel that are susceptible to contamination during dispensing of the therapeutic agent comprise one or more antimicrobial additives which provide antimicrobial efficacy at the applicator and vessel surfaces.

17. The system according to claim 16, wherein:
the applicator comprises a dropper having a cap and a tip;
the antimicrobial additives are distributed on the tip of the dropper and interior surfaces of the cap that are exposed during dispensing of the therapeutic agent; and
the antimicrobial additives have an antimicrobial effect when in physical contact with the therapeutic agent.

18. The system according to claim 16, wherein the antimicrobial additives are distributed on an interior wall of the vessel, the antimicrobial additives providing antimicrobial efficacy at vessel wall surfaces that become exposed as the volume of therapeutic agent within the vessel is reduced due to repeated dispensing over time.

19. A method for dispensing a preservative-free therapeutic agent, comprising:
storing the preservative-free therapeutic agent within a polymeric vessel, the vessel fluidly coupled to a polymeric applicator through which the therapeutic agent is dispensed; and
providing antimicrobial efficacy at surfaces of the applicator that can be contaminated during dispensing of the therapeutic agent.

20. The method according to claim 19, wherein providing antimicrobial efficacy comprises providing antimicrobial efficacy at the applicator surfaces for at least a predetermined number of days and without fouling the therapeutic agent.

21. A method for dispensing a partially-preserved or preserved therapeutic agent, comprising:
storing the partially-preserved or preserved therapeutic agent within a polymeric vessel, the vessel fluidly coupled to a polymeric applicator through which the therapeutic agent is dispensed; and
providing antimicrobial efficacy at surfaces of the applicator and the vessel that can be contaminated during dispensing of the therapeutic agent.

22. The method according to claim 21, wherein providing antimicrobial efficacy comprises providing antimicrobial efficacy at the applicator surfaces for at least a predetermined number of days and without fouling the therapeutic agent.
